(19) United States
(12) Patent Application Publication
(10) Pub. No.: US 2011/0282194 A1
(43) Pub. Date: Nov. 17, 2011

(54) Method and apparatus of quantitative analysis and data mining of medical imaging agent administration

(76) Inventor: Bruce Reiner, Berlin, MD (US)

(21) Appl. No.: 13/067,083

(22) Filed: May 6, 2011

Related U.S. Application Data
(60) Provisional application No. 61/344,009, filed on May 6, 2010.

(51) Int. Cl. A61B 6/00

(52) U.S. Cl. 600/431

(57) ABSTRACT

The present invention focuses on the quantitative data analyses which are derived from the qualitative contrast scorecard data; which is recorded and analyzed through the combined functions (and communication) of the contrast injector and image acquisition (e.g., CT) technologies. This technical data is in turn correlated with the clinical data from the Contrast Scorecard to collectively produce a comprehensive and longitudinal database which tracks all patient, stakeholder, exam, contrast, technology, and institutional data related to the administration of medical imaging contrast agents.

### CROSS-REFERENCE TO RELATED APPLICATIONS

The present application claims priority from U.S. Provisional Patent Application No. 61/344,009, filed May 6, 2010, the contents of which are herein incorporated by reference in their entirety.

### BACKGROUND OF THE INVENTION

1. Field of the Invention

The present invention is related to performing quantitative data analysis derived from the qualitative contrast scorecard data, which is recorded and analyzed through combined functions (and communication) of the contrast injector and image acquisition (e.g., CT) technologies. This technical data is correlated with the clinical data obtained from the contrast scorecard, to collectively produce a comprehensive and longitudinal database which tracks all patient, stakeholder, examination, contrast, technology, and institutional data related to the administration of medical imaging contrast agents.

2. Description of the Related Art

The creation of a comprehensive medical information system database was described in U.S. Pat. No. 7,933,782 to Reiner, which served to collect, store, and analyze a multitude of data associated with the administration of medical imaging contrast agents and provide data-driven analytics related to patient safety, medical economics, quality performance, and clinical outcomes. The data recorded in this database tracked the sequential steps which take place in the collective process of a medical imaging exam, beginning at the time an examination is ordered and ending with clinical management based upon the examination findings. The data is in turn used to objectively analyze clinical performance of each action, along with the various stakeholders who play a role in the collective process.

The data utilized in this invention is primarily qualitative in nature, and is used to create a number of analytics and deliverables, including (but not limited to): 1) patient-specific clinical attributes (related to the risk-benefit analysis of contrast administration); 2) exam appropriateness; 3) comparative safety and clinical efficacy of various contrast agents; 4) individual stakeholder and institutional performance (related to safety, economics, and clinical outcomes); 5) quality control (QC) and quality assurance (QA); 6) end-user education and training; 7) creation of data-driven best practice guidelines; 8) clinical validation and testing of technologies used; 9) computerized decision support; and 10) automated reporting and analysis of adverse clinical outcomes.

However, the prior art does not focus on the quantitative data analyses which are derived from the time-activity curve generated through contrast administration, nor how this data is correlated with the clinical data. Thus, this would provide an important statistic for stakeholders.

### SUMMARY OF THE INVENTION

The present invention focuses on the quantitative data analyses which are derived from the qualitative contrast scorecard data; which is recorded and analyzed through the combined functions (and communication) of the contrast injector and image acquisition (e.g., CT) technologies. This technical data is in turn correlated with the clinical data from the Contrast Scorecard to collectively produce a comprehensive and longitudinal database which tracks all patient, stakeholder, exam, contrast, technology, and institutional data related to the administration of medical imaging contrast agents.

In one embodiment, a computer-implemented method of quantitative analysis of contrast administrations and imaging examinations, includes: recording quantitative data of contrast administration to a patient, over time, from a contrast injector, into a database of a computer system; recording said quantitative data relative to one of an individual organ system, anatomic region, or pathologic finding of the patient, in said database; performing an imaging examination of the patient, using an imaging device and recording additional quantitative data therefrom; coordinating said contrast administration and imaging examination, such that a timing of image acquisition and a relative speed and concentration of contrast administration is performed; and performing an analysis using a processor of said computer system, of differential contrast administration of the patient linked with said recorded quantitative data within said imaging examination, to provide patient safety and imaging protocol information on said contrast delivery and said imaging examination.

In another embodiment, said quantitative data includes a volume and type of contrast, injection rates, and times and sequences of image acquisition.

In yet another embodiment, the method includes using said analysis to provide an optimal imaging examination protocol that is disease-specific, for the patient.

In yet another embodiment, the method includes performing immediate modifications based upon real-time physiologic measurements during said contrast administration and imaging examination.

In yet another embodiment, the method further includes performing auto-modulation of said contrast administration to continuously adjust a volume and rate at which contrast is administered.

In yet another embodiment, the method further includes comparing a plurality of contrast administration protocols to identify a desired time-activity curve related to image quality and safety profile; and creating an optimal imaging examination protocol and said optimal contrast administration protocol therefrom.

In yet another embodiment, the method further includes searching said database to identify data relevant to said optimal imaging examination protocol and said optimal contrast administration protocol.

In yet another embodiment, the method includes recording patient, clinical and contrast-specific qualitative and quantitative data into said database, for different types of pathology specific to different organ system san clinical conditions; searching said database for said clinical and contrast-specific qualitative data; and providing a hierarchical list of pathologic entities for differential diagnosis.

In yet another embodiment, the method includes comparing a pathology-specific contrast time-activity curve with a contrast time-activity curve of non-diseased tissue, to fingerprint contrast pathologies.

In yet another embodiment, the method includes generating a pathologic differential diagnosis based upon said search of said database; and generating a statistical likelihood of each listed diagnostic consideration.

In yet another embodiment, said analysis includes said fingerprint of said contrast pathologies in combination with said quantitative data.

In yet another embodiment, the method includes establishing quality assurance criteria and correlating said with said quantitative data to rate quality assurance at a point of care of the patient.

In yet another embodiment, said quality assurance rating is a quality assurance score, and to achieve a pre-defined measure of quality assurance, said quality assurance score is not to exceed a pre-defined threshold.

In yet another embodiment, the method includes performing a trending analysis of said quality assurance ratings.

In yet another embodiment, the method includes rating an interpretation accuracy of a radiologist with said quality assurance analysis.

In yet another embodiment, the method includes performing data mining to provide optimal contrast strategies for imaging and contrast injector technologies.

In yet another embodiment, the method includes providing data mining to provide interval change in a clinical data of the patient.

In yet another embodiment, the method includes performing data mining to determine a relative safety a diagnostic performance of individual service or institutional providers and their peers.

Thus has been outlined, some features consistent with the present invention in order that the detailed description thereof that follows may be better understood, and in order that the present contribution to the art may be better appreciated. There are, of course, additional features consistent with the present invention that will be described below and which will form the subject matter of the claims appended hereto.

In this respect, before explaining at least one embodiment consistent with the present invention in detail, it is to be understood that the invention is not limited in its application to the details of construction and to the arrangements of the components set forth in the following description or illustrated in the drawings. Methods and apparatuses consistent with the present invention are capable of other embodiments and of being practiced and carried out in various ways. Also, it is to be understood that the phraseology and terminology employed herein, as well as the abstract included below, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the methods and apparatuses consistent with the present invention.

### BRIEF DESCRIPTION OF THE DRAWINGS

FIG. 1 is a schematic diagram of a system for quantifying radiation safety in medical imaging, according to one embodiment consistent with the present invention.

FIG. 2 is a flowchart showing a method of performing an analysis of differential contrast administration linked with recorded quantitative data within an imaging examination, in one embodiment consistent with the present invention.

### DESCRIPTION OF THE INVENTION

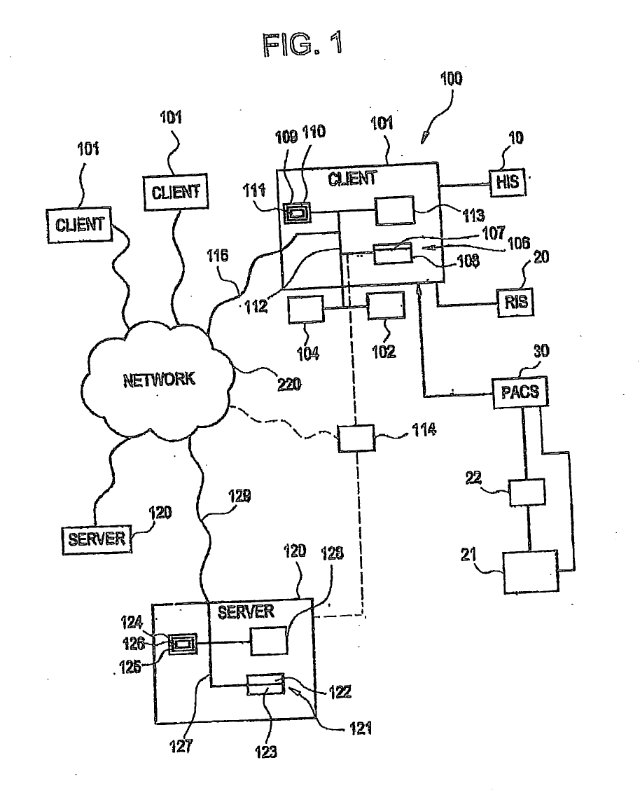

According to one embodiment of the invention illustrated in FIG. 1, medical (radiological) applications may be implemented using the system 100. The system 100 is designed to interface with existing information systems such as a Hospital Information System (HIS) 10, a Radiology Information System (RIS) 20, an acquisition or radiographic device 21, and/or other information systems that may access a computed radiography (CR) cassette or direct radiography (DR) system, a CR/DR plate reader 22, a Picture Archiving and Communication System (PACS) 30, and/or other systems, which are connected to the patient to record certain metrics. The system 100 may be designed to conform with the relevant standards, such as the Digital Imaging and Communications in Medicine (DICOM) standard, DICOM Structured Reporting (SR) standard, and/or the Radiological Society of North America's Integrating the Healthcare Enterprise (IHE) initiative, among other standards.

According to one embodiment, bi-directional communication between the system 100 of the present invention and the information systems, such as the HIS 10, RIS 20, radiographic device, CR/DR plate reader 22, and PACS 30, etc., may be enabled to allow the system 100 to retrieve and/or provide information from/to these systems. According to one embodiment of the invention, bi-directional communication between the system 100 of the present invention and the information systems allows the system 100 to update information that is stored on the information systems. According to one embodiment of the invention, bi-directional communication between the system 100 of the present invention and the information systems allows the system 100 to generate desired reports and/or other information.

The system 100 of the present invention includes a client computer 101, such as a personal computer (PC), which may or may not be interfaced or integrated with the PACS 30. The client computer 101 may include an imaging display device 102 that is capable of providing high resolution digital images in 2-D or 3-D, for example. According to one embodiment of the invention, the client computer 101 may be a mobile terminal if the image resolution is sufficiently high. Mobile terminals may include mobile computing devices, a mobile data organizer (PDA), or other mobile terminals that are operated by the user accessing the program 110 remotely.

According to one embodiment of the invention, an input device 104 or other selection device, may be provided to select hot clickable icons, selection buttons, and/or other selectors that may be displayed in a user interface using a menu, a dialog box, a roll-down window, or other user interface. The user interface may be displayed on the client computer 101. According to one embodiment of the invention, users may input commands to a user interface through a programmable stylus, keyboard, mouse, speech processing device, laser pointer, touch screen, or other input device 104.

According to one embodiment of the invention, the input or other selection device 104 may be implemented by a dedicated piece of hardware or its functions may be executed by code instructions that are executed on the client processor 106. For example, the input or other selection device 104 may be implemented using the imaging display device 102 to display the selection window with a stylus or keyboard for entering a selection.

According to another embodiment of the invention, symbols and/or icons may be entered and/or selected using an input device 104, such as a multi-functional programmable stylus. The multi-functional programmable stylus may be used to draw symbols onto the image and may be used to accomplish other tasks that are intrinsic to the image display, navigation, interpretation, and reporting processes. The multi-functional programmable stylus may provide superior functionality compared to traditional computer keyboard or mouse input devices. According to one embodiment of the invention, the multi-functional programmable stylus also may provide superior functionality within the PACS and Electronic Medical Report (EMR).

According to one embodiment of the invention, the client computer 101 may include a processor 106 that provides client data processing. According to one embodiment of the invention, the processor 106 may include a central processing unit (CPU) 107, a parallel processor, an input/output (I/O) interface 108, a memory 109 with a program 110 having a data structure 111, and/or other components. According to one embodiment of the invention, the components all may be connected by a bus 112. Further, the client computer 101 may include the input device 104, the image display device 102, and one or more secondary storage devices 113. According to one embodiment of the invention, the bus 112 may be internal to the client computer 101 and may include an adapter that enables interfacing with a keyboard or other input device 104. Alternatively, the bus 112 may be located external to the client computer 101.

According to one embodiment of the invention, the image display device 102 may be a high resolution touch screen computer monitor. According to one embodiment of the invention, the image display device 102 may clearly, easily and accurately display images, such as x-rays, and/or other images. Alternatively, the image display device 102 may be implemented using other touch sensitive devices including tablet personal computers, pocket personal computers, plasma screens, among other touch sensitive devices. The touch sensitive devices may include a pressure sensitive screen that is responsive to input from the input device 104, that may be used to write/draw directly onto the image display device 102.

According to another embodiment of the invention, high resolution goggles may be used as a graphical display to provide end users with the ability to review images. According to another embodiment of the invention, the high resolution goggles may provide graphical display without imposing physical constraints of an external computer.

According to another embodiment, the invention may be implemented by an application that resides on the client computer 101, wherein the client application may be written to run on existing computer operating systems. Users may interact with the application through a graphical user interface. The client application may be ported to other personal computer (PC) software, personal digital assistants (PDAs), cell phones, and/or any other digital device that includes a graphical user interface and appropriate storage capability.

According to one embodiment of the invention, the processor 106 may be internal or external to the client computer 101. According to one embodiment of the invention, the processor 106 may execute a program 110 that is configured to perform predetermined operations. According to one embodiment of the invention, the processor 106 may access the memory 109 in which may be stored at least one sequence of code instructions that may include the program 110 and the data structure 111 for performing predetermined operations. The memory 109 and the program 110 may be located within the client computer 101 or external thereto.

While the system of the present invention may be described as performing certain functions, one of ordinary skill in the art will readily understand that the program 110 may perform the function rather than the entity of the system itself.

According to one embodiment of the invention, the program 110 that runs the system 100 may include separate programs 110 having code that performs desired operations. According to one embodiment of the invention, the program 110 that runs the system 100 may include a plurality of modules that perform sub-operations of an operation, or may be part of a single module of a larger program 110 that provides the operation.

According to one embodiment of the invention, the processor 106 may be adapted to access and/or execute a plurality of programs 110 that correspond to a plurality of operations. Operations rendered by the program 110 may include, for example, supporting the user interface, providing communication capabilities, performing data mining functions, performing e-mail operations, and/or performing other operations.

According to one embodiment of the invention, the data structure 111 may include a plurality of entries. According to one embodiment of the invention, each entry may include at least a first storage area, or header, that stores the databases or libraries of the image files, for example.

According to one embodiment of the invention, the storage device 113 may store at least one data file, such as image files, text files, data files, audio files, video files, among other file types. According to one embodiment of the invention, the data storage device 113 may include a database, such as a centralized database and/or a distributed database that are connected via a network. According to one embodiment of the invention, the databases may be computer searchable databases. According to one embodiment of the invention, the databases may be relational databases. The data storage device 113 may be coupled to the server 120 and/or the client computer 101, either directly or indirectly through a communication network, such as a LAN, WAN, and/or other networks. The data storage device 113 may be an internal storage device. According to one embodiment of the invention, the system 100 may include an external storage device 114. According to one embodiment of the invention, data may be received via a network and directly processed.

According to one embodiment of the invention, the client computer 101 may be coupled to other client computers 101 or servers 120. According to one embodiment of the invention, the client computer 101 may access administration systems, billing systems and/or other systems, via a communication link 116. According to one embodiment of the invention, the communication link 116 may include a wired and/or wireless communication link, a switched circuit communication link, or may include a network of data processing devices such as a LAN, WAN, the Internet, or combinations thereof. According to one embodiment of the invention, the communication link 116 may couple e-mail systems, fax systems, telephone systems, wireless communications systems such as pagers and cell phones, wireless PDA's and other communication systems.

According to one embodiment of the invention, the communication link 116 may be an adapter unit that is capable of executing various communication protocols in order to establish and maintain communication with the server 120, for example. According to one embodiment of the invention, the communication link 116 may be implemented using a specialized piece of hardware or may be implemented using a general CPU that executes instructions from program 110. According to one embodiment of the invention, the communication link 116 may be at least partially included in the processor 106 that executes instructions from program 110.

According to one embodiment of the invention, if the server 120 is provided in a centralized environment, the server 120 may include a processor 121 having a CPU 122 or parallel processor, which may be a server data processing device and an I/O interface 123. Alternatively, a distributed CPU 122 may be provided that includes a plurality of individual processors 121, which may be located on one or more machines. According to one embodiment of the invention, the processor 121 may be a general data processing unit and may include a data processing unit with large resources (i.e., high processing capabilities and a large memory for storing large amounts of data).

According to one embodiment of the invention, the server 120 also may include a memory 124 having a program 125 that includes a data structure 126, wherein the memory 124 and the associated components all may be connected through bus 127. If the server 120 is implemented by a distributed system, the bus 127 or similar connection line may be implemented using external connections. The server processor 121 may have access to a storage device 128 for storing preferably large numbers of programs 110 for providing various operations to the users.

According to one embodiment of the invention, the data structure 126 may include a plurality of entries, wherein the entries include at least a first storage area that stores image files. Alternatively, the data structure 126 may include entries that are associated with other stored information as one of ordinary skill in the art would appreciate.

According to one embodiment of the invention, the server 120 may include a single unit or may include a distributed system having a plurality of servers 120 or data processing units. The server(s) 120 may be shared by multiple users in direct or indirect connection to each other. The server(s) 120 may be coupled to a communication link 129 that is preferably adapted to communicate with a plurality of client computers 101.

According to one embodiment, the present invention may be implemented using software applications that reside in a client and/or server environment. According to another embodiment, the present invention may be implemented using software applications that reside in a distributed system over a computerized network and across a number of client computer systems. Thus, in the present invention, a particular operation may be performed either at the client computer 101, the server 120, or both.

According to one embodiment of the invention, in a client-server environment, at least one client and at least one server are each coupled to a network 220, such as a Local Area Network (LAN), Wide Area Network (WAN), and/or the Internet, over a communication link 116, 129. Further, even though the systems corresponding to the HIS 10, the RIS 20, the radiographic device 21, the CR/DR reader 22, and the PACS 30 (if separate) are shown as directly coupled to the client computer 101, it is known that these systems may be indirectly coupled to the client over a LAN, WAN, the Internet, and/or other network via communication links. According to one embodiment of the invention, users may access the various information sources through secure and/or non-secure interne connectivity. Thus, operations consistent with the present invention may be carried out at the client computer 101, at the server 120, or both. The server 120, if used, may be accessible by the client computer 101 over the Internet, for example, using a browser application or other interface.

According to one embodiment of the invention, the client computer 101 may enable communications via a wireless service connection. The server 120 may include communications with network/security features, via a wireless server, which connects to, for example, voice recognition. According to one embodiment, user interfaces may be provided that support several interfaces including display screens, voice recognition systems, speakers, microphones, input buttons, and/or other interfaces. According to one embodiment of the invention, select functions may be implemented through the client computer 101 by positioning the input device 104 over selected icons. According to another embodiment of the invention, select functions may be implemented through the client computer 101 using a voice recognition system to enable hands-free operation. One of ordinary skill in the art will recognize that other user interfaces may be provided.

According to another embodiment of the invention, the client computer 101 may be a basic system and the server 120 may include all of the components that are necessary to support the software platform. Further, the present client-server system may be arranged such that the client computer 101 may operate independently of the server 120, but the server 120 may be optionally connected. In the former situation, additional modules may be connected to the client computer 101. In another embodiment consistent with the present invention, the client computer 101 and server 120 may be disposed in one system, rather being separated into two systems.

Although the above physical architecture has been described as client-side or server-side components, one of ordinary skill in the art will appreciate that the components of the physical architecture may be located in either client or server, or in a distributed environment.

Further, although the above-described features and processing operations may be realized by dedicated hardware, or may be realized as programs having code instructions that are executed on data processing units, it is further possible that parts of the above sequence of operations may be carried out in hardware, whereas other of the above processing operations may be carried out using software.

The underlying technology allows for replication to various other sites. Each new site may maintain communication with its neighbors so that in the event of a catastrophic failure, one or more servers 120 may continue to keep the applications running, and allow the system to load-balance the application geographically as required.

Further, although aspects of one implementation of the invention are described as being stored in memory, one of ordinary skill in the art will appreciate that all or part of the invention may be stored on or read from other computer-readable media, such as secondary storage devices, like hard disks, floppy disks, CD-ROM, a carrier wave received from a network such as the Internet, or other forms of ROM or RAM either currently known or later developed. Further, although specific components of the system have been described, one skilled in the art will appreciate that the system suitable for use with the methods and systems of the present invention may contain additional or different components.

The present invention is directed to the quantitative data (rather than qualitative data) and analytics centered on the contrast administration and derived time-activity curve data of the Contrast Scorecard (described in U.S. Pat. No. 7,933,782 to Reiner, the contents of which are herein incorporated by reference in their entirety). The present invention focuses on the quantitative data analyses analyzed by the program 110 through the combined functions (and communication) of the contrast injector and image acquisition (e.g., CT) technologies. This technical data is in turn correlated by the program 110 with the clinical data contained within the contrast scorecard, to collectively produce a comprehensive and longitudinal database 113, 114 which tracks all patient, stakeholder, exam, contrast, technology, and institutional data related to the administration of medical imaging contrast agents.

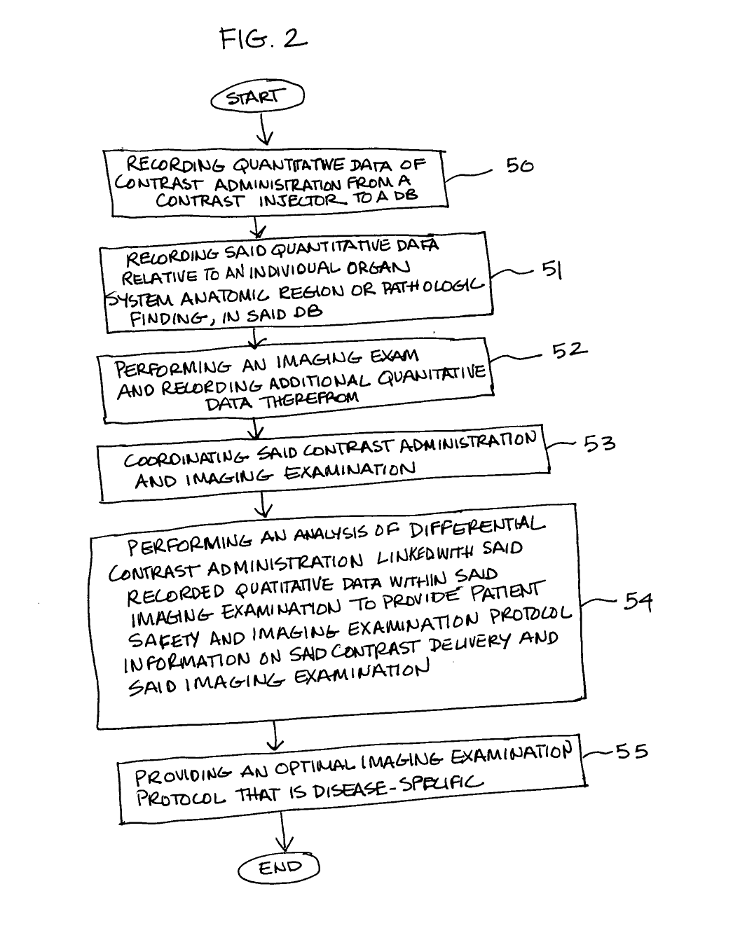

The present invention includes the program 110 essentially plotting the concentration of contrast in a region of concern, over time (see FIG. 2). The derived data from the contrast injector (step 50) can be recorded by the program 110 in the database 113, 114 in both numerical and graphical forms, with the graphical representation (i.e., time-activity curve) stored within the DICOM header for each individual image within the comprehensive imaging dataset. The resulting contrast time-activity analyses can be recorded by the program 110 relative to an individual organ system (e.g., liver), anatomic region (e.g., caudate lobe), or pathologic finding (e.g., liver mass) (step 51). This provides a mechanism for the program 110 to analyze differential contrast administration as it relates to multiple anatomic regions and individual findings within a single imaging examination (step 52). This is particularly relevant when a large area of anatomy is being imaged in a single setting, such as a combined chest, abdomen, and pelvis exam. In addition, the derived time-activity curves are time stamped by the program 110, which provide a mechanism for the program 110 to analyze multi-phase contrast administration (e.g., three phase contrast administration of the liver on CT, during the arterial, portal venous, and hepatic venous phases of enhancement).

One important component of the present invention is the communication between the two principal technologies being used: the contrast injector and the image acquisition technology (e.g., CT scanner). In current practice, these two technologies are separate from one another, so that the operator (i.e., technologist) must manually operate them individually. Injector technologies provide the important technical data required by the program 110 of the present invention. For example, time stamped data can be directly acquired from the contrast injection device that records the type and volume of contrast administered, injection rate, and pressures, and contrast extravasation. These data can be directly correlated by the program 110 with the imaging modality (e.g., CR, MRI) to provide a direct linkage between contrast delivery and the derived imaging data (step 53). New injector technologies have the ability to record these data automatically and link these data with the specific imaging examination (step 54). This provides information on both patient safety as well as the specific imaging protocol employed (step 55).

The most sophisticated method of optimizing contrast administration in current practice consists of bolus tracking, where a small sample dose of contrast is administered, an anatomic region of interest is selected (e.g., right ventricle) and the technologist manually begins image acquisition at the time the contrast bolus reaches the region of interest. This manual workflow and dissociation of the two technologies (i.e., contrast injector and CT scanner) introduces a margin of error and variability which could potentially be circumvented by automation and direct communication between the two technologies.

However, in the present invention, the two technologies would be directly integrated with one another as well as the contrast database 113, 114. The pre-defined anatomic region of interest would be automatically derived by the program 110, based upon the exam type and clinical context.

As an example, in a CT angiogram of the chest for evaluation of pulmonary emboli (i.e., blot clots), the desired region of maximal contrast enhancement would be the pulmonary arteries. During the non-contrast portion of image acquisition, the pulmonary arteries are identified (which can be done manually by the technologist or in an automated fashion using computerized anatomic localization software). As contrast is injected, and as contrast arrival in the pulmonary arteries is detected by the program 110, an automated trigger is initiated by the program 110 between the CT scanner and contrast injector to ensure optimal timing and coordination between contrast administration and image acquisition for the desired region of interest.

As another example, a patient is suspected as having a tumor of the kidney and is referred for an abdominal CT examination. In one case, the protocol used by the user administers the contrast in a standard fashion (injection rate of 2 cc per second for a total of 120 cc of contrast, followed by post-contrast image acquisition. In another case, a more specialized protocol is used which sequentially obtains CT images during different phases of contrast administration (arterial, venous, and excretory phases).

The data recorded in the database 113, 114 by the program 110 would not only identify the volume and type of contrast but also the injection rates and specific times and sequences of image acquisition. This may be important in certain types of diagnoses and overall interpretation accuracy.

This information can also be used by the program 110 to prospectively to determine the optimal imaging/contrast protocol, in accordance with the clinical indication. If, for example, the patient has a suspected renal malignancy, then the program 110 would be able to provide an “optimal” imaging protocol that is disease-specific (based on the scientific literature and established clinical guidelines).

The ability to directly integrate the two technologies has a number of additional advantages, which exceed current practice. Firstly, a number of patient and technology-specific variables can be factored by the program 110 into the process, so that contrast administration is optimized to each individual patient and the technology being employed.

As another example, a patient with cardiac failure (and diminished cardiac output) would have slower blood flow than a patient with normal heart function. As a result, the timing of image acquisition, and relative speed and concentration of contrast administration would require adjustment for image optimization. By the program 110 directly coordinating the injector and acquisition technologies, an automated method of compensation would take place, which can also provide real-time feedback to the injector in adjusting the rate and volume of contrast administered.

Instead of the current practice of static protocols (which call for a pre-determined contrast volume and rate of injection); the present invention would provide for dynamic adjustment of contrast administration by the program 110 based upon in vivo data recorded in the database 113, 114 in the time-activity curve, which in turn is used by the program 110 to modify scanning and injector parameters in real time.

This also provides a mechanism for the program 110 to adjust for differences in technology within a given department. A large imaging department often has several types of CT scanners and injectors, which would each possess its own unique characteristics, relating to the contrast time-activity curve. The ability to make instantaneous “on the fly” adjustments, as described above with respect to the present invention, is essential to contrast optimization; which varies in accordance with individual patient attributes, technology being used, exam type, and clinical context.

While the contrast database 113, 114 provides historical data specific to each individual patient, each patient's medical condition is dynamic and constantly changing. The historical data for each patient can serve as a valuable resource in exam selection, protocol optimization, contrast selection, and dosing parameters. While many of the intrinsic patient attributes remain stable (e.g., body habitus, allergy history), other patient-specific attributes are constantly changing (e.g., cardiac function, state of hydration), which significantly impact decisions related to contrast administration. As a result, while the contrast database 113, 114 serves an important resource for decision support, the ability of the program 110 to make immediate modifications based upon real-time physiologic measurements (i.e., time-activity curve data) is important in improving contrast-related quality and safety.

Another important feature of the present invention, in addition to the integration of the injector and scanning technologies, is the ability of the program 110 to perform “auto-modulation” of contrast administration. This represents the ability for the program 110 to have the integrated acquisition and injector technologies communicate with one another during the process of image acquisition and contrast administration, to continuously adjust the volume and rate in which contrast is administered. This can be dynamically supplemented by data identified by the program 110 within the imaging dataset.

As an example, a patient is undergoing a chest CT angiogram for evaluation of a suspected pulmonary embolism (i.e., blood clot). During the combined image acquisition/contrast injection process, the program 110 identifies when the contrast has entered the right ventricle (which has been defined as the anatomic region of interest).

Once this takes place, the program 110 commands the injector to increase the rate of contrast administration, in order to optimize the opacification of the pulmonary arteries. A second anatomic region of interest (i.e., left atrium) has also been determined by the program 110 to serve as the anatomic marker for discontinuing the contrast bolus.

This is performed because this marks the time in which pulmonary arterial opacification has been completed and the contrast has entered the pulmonary veins, which in turn empty into the left atrium. By using real-time anatomic markers for contrast initiation and contrast termination, the program 110 reduces the total volume of contrast required (which improves patient safety and operational costs), while maximizing the contrast administered during the defined anatomic region of clinical interest (which improves image quality and diagnostic accuracy).

If, for some reason, there is a time delay for the contrast to reach the left atrium (e.g., congestive heart failure), the communication between the injector and CT scanner would ensure that the contrast injection is extended beyond its normal duration, thereby modulating the standard protocol in keeping with the physiology and anatomy of the patient.

This technology integration and auto-modulation feature can be further described in the example of a chest CT angiogram for suspected pulmonary embolism, where the examination was selected due to the symptoms of severe chest pain, which can also be associated with an aneurysm of dissection thoracic aorta or coronary artery obstruction. Normally, the contrast injection would be terminated once contrast reaches the left atrium. However, if thoracic aortic or coronary arterial pathology is of high clinical concern, the injection and scanning protocols would require proactive adjustment to ensure optimal opacification and visualization of the these structures.

While imaging of the thoracic aorta may simply require a slight adjustment in extending the duration of contrast injection, this is not the case of the coronary arteries. In order to optimally visualize the coronary arteries, a number of interventions would be required, including changes in image acquisition speed, slice thickness, and rate of contrast injection.

In order to optimize imaging of the pulmonary arteries, thoracic aorta, and coronary arteries during a single examination, several modifications would have to be selectively made by the program 110 which change acquisition and contrast delivery as each different anatomic region is being analyzed. This can only effectively be accomplished by the program 110 integrating the acquisition and injector technologies with real-time analysis of the time-activity contrast data.

In another example, the pulmonary arteries are the sole focus of the examination, and as a result, the acquisition/injection protocol was tailored by the program 110 specifically to the pulmonary arteries. During the course of the pre-contrast image acquisition (which is customary for all angiographic studies), coronary arterial calcification was identified (by either manual inspection by the CT technologist, or the program's 110 computer-aided detection (CAD) software). The identification of coronary arterial calcification denotes an increased risk of coronary artery disease (i.e., occlusion), which was not initially in the protocol and injection parameters for the chest CT angiogram. Upon recognition by the program 110 however, the auto-modulation feature was enacted by the program 110 to expand the protocol to include evaluation of coronary arterial enhancement.

This makes the program 110 automatically call for a second contrast bolus and change in image acquisition (through the integrated injector and scanning technologies) to be performed, along with real-time calculation of the coronary arterial time-activity curves. The individual time-activity curves of each of the 3 main coronary arteries would then be used to derive an estimate of stenosis by the program 110.

In addition, the time-activity analysis of each coronary artery could be used by the program 110 to select the optimal image processing algorithm for detection of atherosclerotic plaque. This illustrates how the integration of scanner and injector technologies can be used to optimize contrast administration in real time, make “on the fly” adjustments in keeping with anatomic and pathologic states, and utilize the derived time-activity data to derive computerized-measurements related to pathology, and selection of image processing for optimizing image quality and conspicuity of potential pathology.

Computerized program 110 analysis of the time-activity curve data can serve a number of different purposes, including (but not limited to) clinical decision support, education and training, performance analysis, quality assurance, longitudinal data mining, comparative analysis of contrast agents, and creation of new technologies (e.g., image processing software).

A. Clinical Decision Support

While multiple stakeholders are incorporated into the Quality Assurance (QA) Contrast Scorecard, the two principal stakeholders tied to the quantitative time-activity curve analysis are technologists and radiologists. Technologists, who are tasked with image acquisition and processing, can utilize the contrast database 113, 114 to identify the optimal scanning protocol, acquisition parameters, and contrast agent selection and administration, which is specific to the individual patient, examination being performed, technology being utilized, and clinical context (i.e., clinical indication, known pathology).

As an example, a contrast enhanced abdominal CT is ordered on a patient with an indeterminate liver mass recorded on ultrasound. The primary purpose of the CT is to characterize the liver mass in question, provide a pathologic differential diagnosis, and search for potential pathologies in other abdominal viscera. One of the most important features in characterizing the liver mass is its enhancement features, which are determined by the contrast time-activity curve data specific to the mass, relative to the surrounding normal liver parenchyma. An additional feature of importance is identification of the mass's blood supply (i.e., hepatic artery versus portal vein). In order to answer these questions, a triple-phase enhancement protocol is presented by the program 110 to the user, and the user selects same with selective perfusion during the hepatic arterial, portal venous, and hepatic venous phases of contrast enhancement.

One option would be for the technologist to rely on a standard default protocol presented by the program 110, which has been generically created based upon industry recommendations and departmental experience. An alternative approach would be to have the program 110 prompt manual sequential image acquisition after visual observation of the contrast bolus, as it enters the three different phases of enhancement.

The problem with the reliance on a standard default protocol of the first option is that it does not take into account specific patient characteristics (e.g., body habitus, renal function, breath-holding capabilities) and as a result, will create variability in image quality. The principal problem with the second, manual approach is that it is operator dependent, and as a result, will vary according to each individual technologist and their ability to differentiate different phases of hepatic enhancement. In both approaches, little consideration is given as to how the volume and rate of contrast administration is selectively determined to optimize image quality and patient safety.

However, with the present invention, a computer program 110 generated query could be performed to search both the individual and collective patient databases 113, 114. The purpose of the data mining exercise is to identify all data which is relevant to determining the optimal scanning and contrast administration protocols. The various data elements which may be relevant include the following:
1. Patient size, weight, and body mass index
2. Patient allergies
3. Patient renal status and current state of hydration
4. Patient-specific prior contrast agents and corresponding time-activity curves
5. Patient venous accessibility
6. Patient imaging diagnoses (prior report data)
7. Clinical context (liver mass)
8. Patient specific risk factors for liver malignancy (including genetic data)
9. Optimized contrast agent parameters (specific agent, volume, rate of administration) from collective patient contrast database of patients with similar profiles and clinical context.

The rationale for utilizing these data is relatively straightforward. Determination of contrast administration selection and protocol is highly dependent upon the individual patient's attributes, clinical status, and venous accessibility. Venous accessibility (i.e., location and size of intravenous catheter used for contrast administration) is an important determinant in the volume and rate in which contrast can be safely injected. The patient's clinical status (size, renal function, allergies) determines safety factors related to contrast selection and volume. Patient-specific risk factors and prior diagnoses can be used by the program 110 to predict the predisposition to certain disease types, which can be of value in protocol optimization.

Lastly, by the program 110 identifying other patients in the collective database 113, 114 with similar clinical profiles and technologies being used, the program 110 can compare the different contrast administration strategies employed, and identify which had the most desired time-activity curve (image quality) and safety profile (per predetermined profiles). Using these combined data, the program 110 can create an optimized image acquisition/contrast administration protocol, which can be accepted or modified by the technologist.

An example of a modification can be seen when the computer-program 110 generated protocol calls for contrast administration using an 18 gauge intravenous catheter. In the patient being scanned, the intravenous catheter being used is smaller (i.e., 22 gauge) and the patient has a propensity for contrast extravasations due to fragile veins (based upon historical analysis of the contrast database 113, 114). By the user inputting the change in catheter size and concern for extravasation, the program 110 may adjust the contrast administration protocol to accommodate these changes, while still attempting to maximize the contrast time/activity analysis.

Another example of clinical decision support using the time-activity curve data and contrast database 113, 114 can be seen in the image interpretation process. The interpreting radiologist may identify that the time-activity curve data in the region of anatomic interest is relatively flat (i.e., suboptimal). The radiologist may elect to user the program 110 to search the contrast database 113, 114 for image processing techniques best suited for the exam, anatomic region, clinical indication, and time-activity curve profile in question. After the program 110 has identified different image processing techniques which may assist in interpretation, taking into account the specific time-activity curve and clinical data, the radiologist may elect to “apply selected image processing”. The program 110 can then subsequently apply the recommended image processing technique to the imaging dataset, with the goal of improving diagnostic accuracy and reducing the need to inject additional contrast or repeat the examination in question.

A third example of decision support which can be used by both radiologists and clinicians (and also serves as an educational tool) is the use of the contrast enhancement data for differential diagnosis. The manner in which a pathologic process responds to contrast administration can effectively provide important information regarding the intrinsic characteristics of the pathology. Some pathologic conditions (e.g., infection, malignancy) tend to have increased blood supply (as compared to normal tissue) and as a result will demonstrate rapid and vigorous contrast enhancement relative to normal tissue. At the same time, other contrast related data obtained in the contrast time-activity curve (e.g., the rate at which the contrast “washes out” of the pathology in question) can also provide important information as to etiology and malignant potential.

The quantitative data contained within the contrast time-activity curve can therefore, provide important information to assist with diagnosis, and can be used by the program 110 to create a data repository (i.e., database 113, 114) and “fingerprint” of different types of pathology, specific to different organ systems and clinical conditions. This contrast data repository can automatically be queried by the program 110 to provide a hierarchical list of pathologic entities specific to the organ system and anatomic region of interest.

This computer-program 110 generated quantitative contrast analysis can then be cross-referenced by the program 110 with relevant clinical data, specific to the patient (e.g., age, gender, clinical symptoms, genetic and laboratory data, disease problem list, and physical exam findings) to add further specificity and diagnostic accuracy. As discussed in co-pending U.S. patent application Ser. No. 12/659,363, filed Mar. 5, 2010, by Reiner, the contents of which are herein incorporated by reference in their entirety, clinical and imaging data can be cross-referenced by the program 110 to determine what specific data contained within the patient's electronic medical record (EMR) decreases the relative odds of each pathologic entity contained within the computer-program 110 generated differential diagnosis. In the end, the radiologist, clinician, or other healthcare professional can utilize the contrast “fingerprint” database 113, 114 to assist in diagnosis and treatment planning.

B. Contrast Fingerprint

The concept of a contrast “fingerprint” is designed for the program 110 to combine patient, clinical, and contrast-specific qualitative and quantitative data into a hierarchical list of pathology differential diagnosis. Once a specific pathology is identified by the program 110 in the medical imaging being performed, a series of repetitive images are obtained over the anatomic region of interest, thereby creating a pathology-specific contrast time-activity curve, which can be compared by the program 110 with the contrast time-activity curve of the surrounding “normal” tissue (which serves as a reference, allowing for the unique technical attributes of the contrast injection). The program 110 superimposes the “pathology” and “normal” anatomic-specific time activity curves and derives a list of contrast quantitative analyses from the pathology, which can serve as a means to categorize pathologies according to their contrast “fingerprints”.

In addition to the contrast quantitative data used in determining pathologic diagnosis, a number of other variables would be included in the analysis by the program 110, in order to provide increased diagnostic specificity, related to technical, patient, and clinical data. The data elements included by the program 110 in the comprehensive contrast “fingerprint” analysis would include (but not be limited to) the following:
1. Patient demographics (e.g., age, gender, ethnicity).
2. Patient genetic profile (e.g., genomic and proteomic data specific to the individual patient, which predicts predisposition to specific disease states).
3. Clinical data (e.g., laboratory data, disease problem lists, risk factors, symptoms).
4. Anatomy (e.g., specific anatomic region and/or organ system of interest).
5. Medical imaging data (e.g., previously documented imaging data from historical reports; including findings, temporal change, response to medical intervention).
6. Medical device data (e.g., modality and technical attributes of the image acquisition device).
7. Contrast administration data (quantitative data derived from the contrast time-activity curves, which track contrast uptake and washout over time. In addition to the time-activity curve data, contrast-related data would include the contrast agent, injector technology, volume and rate of contrast delivery).

The analysis by the program 110 would begin by utilizing the quantitative time-activity curve data to classify the pathology according to the statistical likelihood of disease (i.e., normal versus pathologic), the category of the disease state (e.g., neoplasm, infection), and the specific sub-type of pathology (e.g., hepatic adenoma, hepatocellular carcinoma). Given the specific anatomic region of interest, medical device, and contrast technical data inputs, a computer program 110 search of the contrast time-activity database can be performed (in a manner similar to a computerized query of a fingerprint database). The cumulative imaging/contrast data are then used by the program 110 to identify similar entries in the database 113, 114 which match the anatomic, imaging, and contrast profiles of the case in question.

The search analytics by the program 110 can be expanded to also include patient-specific demographic, genetic, and clinical data if desired, in an attempt to increase the specificity of the differential diagnosis. Artificial intelligence techniques (e.g., neural networks) used by the program 110 can subsequently cross-reference the collective data (with an emphasis on the contrast quantitative data) to produce a program 110 generated pathologic differential diagnosis, with an individual statistical likelihood of each listed diagnostic consideration. The end-user has the option to modifying this list by selectively prioritizing or editing out individual data elements for differential weighting.

The net result of the analytics is a hierarchical (i.e., rank order) list of pathologic differential diagnosis, based upon the computer program 110 analysis of the contrast and related clinical data. The end-user can review individual pathologic options in more detail by activating the entity of interest on screen, which the program 110 then presents in more detail as information for review, including (but not limited to) the contrast time-activity curve, technical variables, and individual clinical data. If desired, a side-by-side comparison or superimposition of the contrast time-activity curves can be displayed by the program 110 to directly visualize and compare the “unknown” and “documented” pathologies.

An important feature of the contrast “fingerprint” database 113, 114 is the correlation by the program 110 with established pathologic diagnoses, which can be done by longitudinal analysis of the patient electronic medical record (EMR). Data sources for used by the program 110 for establishing pathologic diagnosis include (but are not limited to) pathology reports, discharge summaries, treatment response, serial imaging report data, and surgical/procedural notes. This provides a comprehensive contrast database 113, 114 with established pathologic correlation to facilitate clinical decision support at the point of care, along with unique education/training opportunities, as described below.

C. Education and Training

The quantitative data derived by the program 110 from the contrast time-activity analysis yields important information, which can assist in diagnosis and treatment planning. However, the technical data associated with the contrast administration is equally important in the overall analysis. As an example, the rate and magnitude at which contrast enhancement takes place within a given anatomic structure or pathologic process will be dependent upon the specific contrast agent used, volume and rate of contrast administration, and location and size of the catheter used. This “technical” data would therefore, is combined by the program 110 with the “fingerprint” data of the contrast time-activity curve to obtain an accurate and reproducible analysis.

With respect to clinical decision support, the application of this quantitative “fingerprint” data by the program 110 to pathology differential diagnosis shows how the Contrast Scorecard database 113, 114 could be used to generate a computer program 110 derived differential diagnosis. An end-user (e.g., radiology resident, medical student) could also use this database 113, 114 to study the different contrast “fingerprints” of different types of pathology. If, for example, the end-user wanted to understand how different types of liver lesions can be characterized based upon their contrast “fingerprint”, they could either enter the organ system/anatomic region of interest or a specific pathologic diagnosis. The program 110 could then provide the end-user with a number of contrast “fingerprints” fulfilling the search criteria. In addition, the program 110 could also show different presentations of contrast “fingerprints” for similar pathologies, with associated technical contrast data which accounts for differences in the contrast fingerprint.

To illustrate how this educational application might be used, the example of a radiology or surgical resident who is interested in learning more about different contrast enhancement patterns of different types of liver lesions, follows. The various steps and options employed, follow:

Step 200: Resident signs into Contrast Database using Biometrics for authentication/identification, and the program 110 validates the identification.

Step 201: Resident is presented by the program 110 with a number of functions, of which he/she selects the Education/Training option.

Step 202: After the Education module is opened by the program 110, the resident selects the education program of interest, which in this case is “Contrast Fingerprint Analysis”, and the program 110 opens the selection.

Step 203: The resident then selects the Search option, and the program 110 presents a number of query options to narrow the search parameters, including (but not limited to) organ system, anatomic region, pathology, disease states, and contrast agent.

Step 204: The resident selects the “Pathology” option, and after the program 110 opens this selection, the resident can either manually insert the pathology of interest or select from an itemized list presented by the program 110.

Step 205: For example, the resident inputs the pathology of interest “Hepatocellular Carcinoma”, and the program 110 opens this selection.

Step 206: The program 110 provides a number of additional options to narrow the search parameters (e.g., patient profile data, institutional demographics, contrast agent characteristics, technology utilized, pathology sub-type etc.), and the resident selects which criteria are of interest.

Step 207: The program 110 opens the criteria of interest and then queries the available Contrast Scorecard databases 113, 114 (which could be institutional, local, regional, national, or international).

Step 208: Relevant examples of contrast “fingerprints” which fulfill the search criteria are presented by the program 110 to the resident.

Step 209: The resident can select the individual contrast “fingerprints” of record, and those which have been activated are presented by the program 110 along with relevant clinical and technical data.

Step 210: The resident can subsequently elicit additional searches using the program 110 commands, based upon specific clinical and/or technical data.

Step 211: As each selection is made by the resident, an updated list of contrast “fingerprints” are presented for review by the program 110.

For example, if the resident was to select “Technical Data”, he/she would be presented by the program 110 with all of the pertinent technical information regarding that particular “fingerprint” (e.g., contrast agent, injector/scanning technologies used, volume, rate, and duration of contrast administration, size and location of intravenous catheter, etc.).

In another example, if the resident was to select the “Clinical Data” option, he/she would be presented by the program 110 with numerous clinical data specific to the patient in whom the “fingerprint” was derived (e.g., age, gender, genetic data, laboratory data, pathology grade, treatment, etc.).

In addition to “clinical” education/training (e.g., pathology), the present invention can also be used to facilitate “technical” education and training for technologists regarding the technical aspects of the contrast time-activity curve. In this application, the program 110 can be used to learn how the various technical aspects of contrast administration influence disease detection, image quality, and patient safety.

In an exemplary embodiment, the diagnostic efficacy of a chest CT angiogram for the diagnosis of pulmonary embolism is predicated upon the selective opacification of the pulmonary arteries (i.e., anatomic region of interest), while patient safety is predicated upon the administration of the lowest possible volume of contrast required for definitive diagnosis. Technologists could utilize the program 110 to review different examples of contrast fingerprints and their corresponding images, based upon different technical input data. Along with the combined images and contrast “fingerprints”, the educational module of the program 110 could show how changes in different technical input parameters (e.g., contrast volume, rate of injection, catheter size) could change the contrast fingerprint, resulting images, and degree of pulmonary arterial opacification. As a result, in addition to the program's 110 ability to query the Contrast Scorecard database 113, 114 for relevant examples, the database 113, 114 can be used by the program 110 to create a simulation device which shows how image quality and the contrast “fingerprint” changes as the different technical variables are modified. In this simulation example, a technologist could select from a number of options (e.g., Contrast Agent) and see how the images, pulmonary arterial opacification, and fingerprint are modified by the program 110 as different contrast agents are selected.

D. Quality Assurance

In current procedures, the practice of quality assurance (QA) as it relates to image quality and contrast administration is largely left up to each individual institution and technologist performing the imaging exam in question. While one technologist may be extremely vigilant regarding image quality and contrast enhancement, another may be more lax. The end result is that QA tends to be highly variable and subjective in nature.

The quantitative data contained within the Contrast Scorecard of the present invention provides an objective, easy to use, and reproducible means for QA as it relates to image quality and contrast administration. A set of standardized QA criteria can be established by the program 110, which in turn is correlated by the program 110 with the derived quantitative data to rate QA at the point of care, offering an immediate objective measure of image quality and opportunity for improvement, in the event of a significant QA deficiency.

In the exemplary embodiment of the CT angiography of the patient's chest for evaluation of pulmonary emboli, the standardized data for measuring contrast and image quality QA, requested by the program 110, could include any of the following metrics:
1. Degree of opacification within the main pulmonary artery in Hounsefield units (HU).
2. Ratio of main pulmonary artery to thoracic aorta opacification.
3. Ratio of main pulmonary artery to pulmonary vein opacification.

Using the defined criteria (e.g., opacification of main pulmonary artery in HU), the program 110 would generate a standardized calculation, with a pre-defined image quality/contrast score, and present this to the performing technologist. If the resulting QA score exceeds a pre-defined departmental threshold, no further action is required by the technologist. If, on the other hand, the pre-defined threshold is not achieved, as determined by the program 110, the technologist is presented by the program 110 with a number of options:
1. Submit exam “as is”.
2. Perform additional sequence of images with additional contrast bolus.
3. Repeat exam in 24 hours.
4. Order an alternative imaging exam.
5. Consult radiologist.

Upon presentation by the program 110 of the options, the technologist selects his/her preferred option and enters any additional data for consideration (e.g., equipment malfunction, uncooperative patient). This data is then recorded by the program 110 in the Contrast Scorecard QA database 113, 114, which can be longitudinally analyzed using the program 110 (by supervisory technologist, department administrator, or chief radiologist) to assess QA trending as it relates to patients, technologists, technology, and contrast.

If the technologist elects to perform an additional sequence (with an additional contrast bolus) the program 110 will search the patient's clinical data (e.g., renal status, state of hydration, size) to determine the relative safety associated with additional contrast, and make a recommendation for optimizing quality and safety (which is another clinical decision support feature of the present invention).

In addition to the computer program 110 generated QA scoring analysis, the radiologist and/or clinicians reviewing the imaging dataset are also presented by the program 110 with the option of subjectively grading image quality and contrast administration relative to the clinical context and diagnostic accuracy of the examination performed. This data is also entered by the program 110 into the Contrast Scorecard QA database 113, 114 for analysis. The purpose of the combined QA analysis is to standardize QA using objective and reproducible quantitative data, while correlating this data with patient safety and physician quality perceptions.

In addition to the program 110 generating an objective QA score based upon the pre-defined quality metrics, the Contrast Scorecard database 113, 114 can also serve as an educational aide to the technologist by showing them how alteration of different technical contrast parameters could improve the QA scores (Education function).

In an exemplary embodiment, in the example of the chest CT angiogram performed for pulmonary emboli, the examination has resulted in a suboptimal QA score. After the QA analysis has been completed by the program 110, the program 110 makes recommendations as to how technical parameters could be modified to improve the QA score for the patient in question and technology being used. Along with these recommendations, the program 110 could show how the contrast time-activity curves (i.e., fingerprints) would be modified with the recommended changes, along with the resulting changes in image quality and pulmonary artery opacification. This would be another example of a computer simulation where the program 110 would utilize the actual QA data and historical QA database 113, 114 to create technical improvement options and visually demonstrate how these would translate into improved contrast time-activity curves and image quality.

Another example of how the computer program 110 generated contrast QA could be used to improve clinical outcomes would be the correlation by the program 110 of individual radiologist interpretation accuracy with the computer program 110 generated QA analysis. By understanding how subtle variations in image/contrast QA effect radiologist performance (i.e., diagnostic accuracy, confidence of diagnosis) for a given exam type and clinical context, a profile can be derived by the program 110 for each individual radiologist. This information can be factored by the program 110 into the QA analysis at the time of image capture and used to guide appropriate intervention.

In the example of the QA deficient CT angiogram of the chest, the technologist can be presented by the program 110 with an historical QA record of the available radiologist(s) to assist with determining the best course of action. For example, the sole radiologist interpreting CT chest angiography at this particular time may have a relatively poor performance record relating to that particular exam type and QA score. This may prompt the technologist to perform an additional sequence, repeat the examination at a later date, or hold the examination for interpretation by another radiologist. This is not intended to be punitive, but instead match the relative QA strengths and deficiencies of all parties, with the aim of improved healthcare outcomes.

E. Performance Analysis

The data contained within the Contrast Scorecard database 113, 114 provides an objective means with which all stakeholders and variables involved in the collective process can objectively be analyzed by the program 110 for performance evaluation (see U.S. patent application Ser. No. 12/010,707, as noted above). The ability of the program 110 to extend this analysis to the quantitative data derived from the contrast time-activity curve provides additional depth to the analysis, particularly with respect to the contrast agents and technologies being used. This can be used by the program 110 to optimize contrast agent selection, specific to the individual patient, exam being performed, and technology in use. As new contrast agents or technologies are introduced, the Contrast Scorecard database 113, 114 can provide valuable and objective data relating the quality, safety, and cost efficacy of these new interventions relative to their predecessors. This provides a unique mechanism to objectively gauge incremental improvement relating to contrast administration.

F. Data Mining

A large and diverse number of elements are contained within the Contrast Scorecard database 113, 114 such that the program 110 can track, record, analyze, and cross-reference data related to the patient, clinical context, contrast agent, injection parameters, and imaging/enhancement characteristics of normal and abnormal anatomy. The standardized data collected within individual Contrast databases 113, 114 can be co-mingled with similar databases such that the program 110 can create a mechanism for departmental, institutional, local, regional, national, and international analysis.

Patient and disease-specific contrast profiles can be derived by the program 110 from longitudinal analysis of these databases 113, 114, which serve as an important mechanism of real-time electronic notification of contrast-related risk, optimal use, and iatrogenic complications. These data-driven prompts are derived by the program 110 from the individual patient's contrast and clinical history, as well as those patients with similar profiles. The contrast-related data mining exercise begins at the time of order entry, at which time examination and contrast appropriateness is evaluated by the program 110. Based upon the clinical data presented (e.g., clinical indication), the database 113, 114 can be queried by the program 110 to determine whether the examination requested is appropriate and whether the patient is a candidate for contrast administration. Relevant data elements would include the patient's past contrast history, allergies, renal/cardiac function, and clinical context. If any contraindication of increased risk is identified, the information is presented to the ordering physician by the program 110 along with recommended alternatives.

In an exemplary embodiment, prior to performance of the ordered examination, the patient and all service providers (e.g., technologist, nurse) undergo registration and authentication using Biometrics, in step 300.

At that time, the patient's contrast and clinical databases 113, 114 are queried by the program 110, in step 301, to optimize examination selection, protocol, and contrast decisions.

Based upon available data within the individual patient and collective Contrast databases 113, 114, intelligent recommendations are presented to the staff by the program 110 in step 302, relating to optimal contrast strategies for the imaging and contrast injector technologies being used, clinical context, and patient-specific attributes.

The specific patient attributes used to determine “best practice” recommendations would include (but not be limited to) size, age, physiology (i.e., cardiac and renal function), state of hydration, allergies, prior contrast history (including complications), and venous access.

The clinical attributes would include the patient's medical history (e.g., disease problem list), clinical indication for the examination, genetic predisposition, and suspected pathology.

The technical attributes would include the specific imaging and injector technologies being used, along with the catheter location and gauge (i.e., size).

An additional data mining feature to optimize contrast administration would include the historical image data, which would include past findings documented in imaging reports, their enhancement characteristics, and quality assurance (QA) related contrast issues. As an example, if a patient is having a follow-up chest CT angiogram for pulmonary embolism, the imaging database 113, 114 would be searched by the program 110 to determine what findings were reported, the technical factors related to contrast administration, and the recorded image quality.

If, for example, two prior chest CT angiograms were performed in the same patient (without interval change in renal and cardiac function), the database 113, 114 query by the program 110 would determine which of the two studies was found to have the higher image quality scores, and the program 110 would present these injection parameters as the default for current use.

If, on the other hand, the QA analysis by the program 110 of the prior study identified QA deficiencies (e.g., contrast agent used, volume, or rate of contrast delivery); the program 110 may make amended recommendations for the current study based upon historical analysis of the imaging database 113, 114.

The data mining deliverables could also include a program 110 analysis of interval change in the patient's clinical data. For example, in the event the patient's renal status has deteriorated since the time of the prior CT angiogram by 30% (based upon laboratory data such as BUN, creatinine, or glomerular filtration rate (GFR)), the program 110 can present the technologist with modified contrast administration recommendations based upon modification of the prior contrast data and/or search of the comprehensive contrast database 113, 114 to identify patients with similar clinical profiles, imaging studies, and technologies being used. The databases 113, 114 of these comparable patients would then be queried by the program 110 to determine the optimal contrast selection parameters and present these at the time of protocol determination.

The end result of this proactive data mining by the program 110 for clinical decision support is that the contrast database 113, 114 becomes “self-learning’ and the derived data mining recommendations by the program 110 become iterative in nature. As new data is recorded by the program 110 in the database 113, 114 and cross-referenced with historical contrast data, refinements in contrast recommendations and decision is made by the program 110; both in support of protocol optimization (at the level of the technologist,) and diagnosis (at the levels of the radiologist and clinician). At the same time, the ability of the program 110 to automate QA (as it relates to image quality, contrast optimization, and diagnostic accuracy) collectively creates a mechanism to automate feedback to clinical end-users and technology producers as to the relative success/deficiencies of their services and products. The ultimate goal is to improve healthcare outcomes, through enhanced patient safety and improved diagnosis, while also improving medical economics.

The longitudinal analysis of the Contrast database 113, 114 also provides a mechanism for determining best practice (i.e., evidence-based) medical guidelines; specific to the individual patient profile, clinical context, and technology in use.

The data mining by the program 110 can also be directed to individual service or institutional providers to determine how their relative safety and diagnostic performances relate to their peers. This information is not intended to be punitive in nature, but instead serve as an educational guide to opportunity for improvement. Repetitive outliers can in turn be identified and subjected to more rigorous oversight and educational requirements, in an attempt to improve performance. At the same time, contrast and technology providers can utilize the objective data within the Contrast database 113, 114 to contrast and compare the relative safety and quality performance of their products relative to their peers.

In sum, the quantitative data and derived analyses of the present invention provides an objective mechanism for improving patient safety, image quality, and diagnostic accuracy related to medical imaging. One of the unique attributes of the present invention is the direct integration of the image acquisition and contrast injector technologies, which collectively record, track, and analyze contrast and imaging data in tandem. This also provides an effective mechanism for instituting immediate and real-time protocol adjustments, which is currently not feasible in current practice, where the two technologies are disparate. The quantitative contrast analysis has a number of unique deliverables including (but not limited to) automated quality assurance (related to contrast optimization and image quality), computerized diagnosis (related to the contrast enhancement profile (i.e. “fingerprint”) of a given pathology), and data-driven clinical decision support, education/training, and performance analysis.

It should be emphasized that the above-described embodiments of the invention are merely possible examples of implementations set forth for a clear understanding of the principles of the invention. Variations and modifications may be made to the above-described embodiments of the invention without departing from the spirit and principles of the invention. All such modifications and variations are intended to be included herein within the scope of the invention and protected by the following claims.

What is claimed is:

1. A computer-implemented method of quantitative analysis of contrast administrations and imaging examinations, comprising:
recording quantitative data of contrast administration to a patient, over time, from a contrast injector, into a database of a computer system;
recording said quantitative data relative to one of an individual organ system, anatomic region, or pathologic finding of the patient, in said database;
performing an imaging examination of the patient, using an imaging device and recording additional quantitative data therefrom;
coordinating said contrast administration and imaging examination, such that a timing of image acquisition and a relative speed and concentration of contrast administration is performed;
performing an analysis using a processor of said computer system, of differential contrast administration of the patient linked with said recorded quantitative data within said imaging examination, to provide patient safety and imaging examination protocol information on said contrast delivery and said imaging examination.

2. The method of claim 1, wherein said quantitative data includes a volume and type of contrast, injection rates, and times and sequences of image acquisition.

3. The method of claim 2, further comprising:
using said analysis to provide an optimal imaging examination protocol that is disease-specific, for the patient.

4. The method of claim 1, further comprising:
performing immediate modifications based upon real-time physiologic measurements during said contrast administration and imaging examination.

5. The method of claim 4, further comprising:
performing auto-modulation of said contrast administration to continuously adjust a volume and rate at which contrast is administered.

6. The method of claim 3, further comprising:
comparing a plurality of contrast administration protocols to identify a desired time-activity curve related to image quality and safety profile; and
creating an optimal imaging examination protocol and said optimal contrast administration protocol therefrom.

7. The method of claim 6, further comprising:
searching said database to identify data relevant to said optimal imaging examination protocol and said optimal contrast administration protocol.

8. The method of claim 7, further comprising:
recording patient, clinical and contrast-specific qualitative and quantitative data into said database, for different types of pathology specific to different organ system san clinical conditions;
searching said database for said clinical and contrast-specific qualitative data; and
providing a hierarchical list of pathologic entities for differential diagnosis.

9. The method of claim 8, further comprising:
comparing a pathology-specific contrast time-activity curve with a contrast time-activity curve of non-diseased tissue, to fingerprint contrast pathologies.

10. The method of claim 8, further comprising:
generating a pathologic differential diagnosis based upon said search of said database; and
generating a statistical likelihood of each listed diagnostic consideration.

11. The method of claim 10, wherein said analysis includes said fingerprint of said contrast pathologies in combination with said quantitative data.

12. The method of claim 1, further comprising:
establishing quality assurance criteria and correlating said with said quantitative data to rate quality assurance at a point of care of the patient.

13. The method of claim 12, wherein said quality assurance rating is a quality assurance score, and to achieve a pre-defined measure of quality assurance, said quality assurance score is not to exceed a pre-defined threshold.

14. The method of claim 13, further comprising:
performing a trending analysis of said quality assurance ratings.

15. The method of claim 13, further comprising:
rating an interpretation accuracy of a radiologist with said quality assurance analysis.

16. The method of claim 14, further comprising:
performing data mining to provide optimal contrast strategies for imaging and contrast injector technologies.

17. The method of claim 16, further comprising:
providing data mining to provide interval change in a clinical data of the patient.

18. The method of claim 17, further comprising:
performing data mining to determine a relative safety an diagnostic performance of individual service or institutional providers and their peers.

19. A computer readable medium containing executable code for performing quantitative analysis of contrast administrations and imaging examinations, comprising:
recording quantitative data of contrast administration to a patient, over time, from a contrast injector, into a database of a computer system;
recording said quantitative data relative to one of an individual organ system, anatomic region, or pathologic finding of the patient, in said database;
performing an imaging examination of the patient, using an imaging device and recording additional quantitative data therefrom;
coordinating said contrast administration and imaging examination, such that a timing of image acquisition and a relative speed and concentration of contrast administration is performed; and
performing an analysis using a processor of said computer system, of differential contrast administration of the patient linked with said recorded quantitative data within said imaging examination, to provide patient safety and imaging protocol information on said contrast delivery and said imaging examination.

20. A computer system for performing quantitative analysis of contrast administrations and imaging examinations, comprising:
at least one memory which contains at least one program which comprises the steps of:
recording quantitative data of contrast administration to a patient, over time, from a contrast injector, into a database of a computer system;
recording said quantitative data relative to one of an individual organ system, anatomic region, or pathologic finding of the patient, in said database;
performing an imaging examination of the patient, using an imaging device and recording additional quantitative data therefrom;
coordinating said contrast administration and imaging examination, such that a timing of image acquisition and a relative speed and concentration of contrast administration is performed; and
performing an analysis using a processor of said computer system, of differential contrast administration of the patient linked with said recorded quantitative data within said imaging examination, to provide patient safety and imaging protocol information on said contrast delivery and said imaging examination; and
at least one processor for executing the program.
